(19) United States
(12) Patent Application Publication
(10) Pub. No.: US 2007/0197913 A1
(43) Pub. Date: Aug. 23, 2007

(54) Ultrasonic scanning apparatus and method for diagnosing bladder

(75) Inventors: Jung-Hoe Kim, Seoul (KR); Seung-Tai Kim, Gwacheon-si (KR)

Correspondence Address: THE FARRELL LAW FIRM, P.C., 333 EARLE OVINGTON BOULEVARD, SUITE 701, UNIONDALE, NY, 11553, US

(73) Assignee: Mcube Technology Co., Ltd., Seoul (KR)

(21) Appl. No.: 11/651,186

(22) Filed: Jan. 9, 2007

(30) Foreign Application Priority Data
Jan. 9, 2006 (KR) 10-2006-002257

(51) Int. Cl. A61B 8/00

(52) U.S. Cl. 600/447

(57) ABSTRACT

Disclosed herein is ultrasonic scanning apparatus and method for diagnosing a bladder. The ultrasonic scanning apparatus includes a transducer, a transducer support, an analog signal processing unit, a display unit, a central control unit, a first stepping motor, a second stepping motor, and a drive control unit. The transducer emits ultrasonic signals for respective scan lines and receives ultrasonic signals reflected from an object. The transducer support configured such that the transducer is fixedly installed therein. The analog signal processing unit converts the ultrasonic signals into digital signals. The display unit outputs specific image signals. The central control unit performs image processing on the digital ultrasonic signals, outputs the results of the processing to the display unit, and controls the overall operation of the apparatus. The first stepping motor rotates in a first direction. The second stepping motor rotates in a second direction. The drive control unit controls the operation of the first and second stepping motors in response to drive control signals provided from the central control unit.

### PRIORITY

This application claims priority under 35 U.S.C. §119 to an application filed with the Korean Industrial Property Office on Jan. 9, 2006, assigned application serial number 10-2006-0002257, the contents of which are incorporated herein by reference.

### BACKGROUND OF THE INVENTION

1. Field of the Invention

The present invention relates generally to a portable ultrasonic scanning apparatus for diagnosing a bladder and an ultrasonic scanning method using the apparatus and, more particularly, to a portable and small-sized ultrasonic scanning apparatus, which can automatically measure the amount of urine in the bladder, and an ultrasonic scanning method, which can measure the amount of urine in the bladder using the apparatus.

2. Description of the Related Art

Generally, an ultrasonic system is a system that emits ultrasonic signals to an object to be examined using the piezoelectric effect of a transducer, receives the ultrasonic signals reflected from the discontinuous planes of the object, converts the received ultrasonic signals into electrical signals, and outputs the electrical signals to a predetermined display device, thus enabling examination of the internal states of the object. Such an ultrasonic system is widely used for medical diagnosis equipment, non-destructive testing equipment or underwater detection equipment.

However, most conventional ultrasonic scanning apparatuses are inconvenient in that they cannot be easily carried due to their large size and heavy weight. To solve the inconvenience, various portable ultrasonic scanning apparatuses have been proposed. Korean Utility Model registration No. 20-137995 discloses a “Portable ultrasonic scanning apparatus.”

Meanwhile, when examining bladder abnormalities or urinary difficulty, measuring the amount of urine is an essential procedure. Furthermore, prior to urination using a catheter, the amount of urine in a bladder should be measured to account for urine that may be retained after the operation. In addition, in urination training, the amount of urine in a bladder should be measured as a guideline.

Various types of ultrasonic scanning equipment may be used to measure the amount of urine in a bladder as described above. In this case, two methods are used. A first method calculates the amount of urine from respective ultrasonic images for a perpendicular plane and a horizontal plane, which are obtained using typical ultrasonic scanning equipment. However, although many algorithms has been proposed and used for the method, the first method is problematic in that it not only exhibits a considerable error rate but also exhibits different results for different users. A second method uses dedicated ultrasonic equipment for measuring the amount of urine. U.S. Pat. No. 4,926,871 discloses dedicated ultrasonic equipment. However, the dedicated ultrasonic equipment based on the second method has a disadvantage in that it also calculates the amount of urine chiefly using two ultrasonic images that are related to the perpendicular and horizontal planes of a bladder, respectively, and in that a user must find the area indicating the greatest size and select it in order to calculate of the amount of urine.

Accordingly, the present applicant proposes a method of accurately calculating the amount of urine in a bladder while minimizing user interference.

### SUMMARY OF THE INVENTION

Accordingly, the present invention has been made keeping in mind the above problems occurring in the prior art, and an object of the present invention is to provide an ultrasonic scanning apparatus for diagnosing a bladder, which can accurately calculate the amount of urine in the bladder while minimizing user interference.

Another object of the present invention is to provide an ultrasonic scanning apparatus for diagnosing a bladder, which has a size and a weight suitable for portable applications.

A further object of the present invention is to provide an ultrasonic scanning method, which, in the ultrasonic scanning apparatus, can accurately measure the amount of urine in the bladder using received ultrasonic signals.

In order to accomplish the above objects, the present invention provides an ultrasonic scanning apparatus for diagnosing a bladder, including a transducer for emitting ultrasonic signals for respective scan lines, and receiving ultrasonic signals reflected from an object; a transducer support configured such that the transducer is fixedly installed therein; an analog signal processing unit for converting the ultrasonic signals, which are transmitted from the transducer, into digital signals; a display unit for outputting specific image signals; a central control unit for performing image processing on the digital ultrasonic signals transmitted from the analog signal processing unit, outputting the results of the processing to the display unit, and controlling the overall operation of the apparatus; a first stepping motor for rotating in a first direction; a second stepping motor for rotating in a second direction; and a drive control unit for controlling the operation of the first and second stepping motors in response to drive control signals provided from the central control unit; wherein the transducer support rotates in the first direction as the first stepping motor rotates, the transducer support rotates in the second direction as the second stepping motor rotates, and the central control unit calculates the amount of urine in the bladder using a plurality of pieces of ultrasonic information about n scan lines for each of m planes of the bladder, which are sequentially received from the analog signal processing unit.

The central control unit detects the locations of front and rear walls using the ultrasonic information about n scan lines for each of m planes, obtains difference values corresponding to differences between the detected locations of the front and rear walls for the scan lines, obtains an area for the image of a corresponding plane using the difference values, obtains correction coefficients for respective planes, calculates radii using areas for the images of respective planes, and calculating corrected radii by applying the correction coefficients to the calculated radii, obtains the average radius of the corrected radii for the respective planes, and obtains a total volume using the average radius.

In addition, an ultrasonic diagnosis method, the ultrasonic diagnosis method measuring the amount of urine in a bladder by sequentially receiving ultrasonic information about n scan lines for each of m planes of the bladder from the transducer of an ultrasonic scanning apparatus, the ultrasonic diagnosis method including the steps of (a) detecting the locations of front and rear walls using the ultrasonic information about n scan lines for each of m planes; (b) obtaining difference values between the detected locations of the front and rear walls for the respective scan lines; (c) obtaining an area for the image of a corresponding plane using the difference values; (d) obtaining correction coefficients for respective planes; (e) calculating radii using areas for the images of respective planes, and calculating corrected radii by applying the correction coefficients to the calculated radii; (f) obtaining the average radius of the corrected radii for the respective planes; and (g) obtaining a total volume using the average radius.

The step (d) includes (d-1) detecting the maximum of the difference values for the respective scan lines for each plane; (d-2) obtaining the greatest of the maximum values for the respective planes; and (d-3) obtaining a correction coefficient for each plane using a ratio of the maximum value for each plane to the greatest of the maximum values.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a block diagram schematically showing the internal construction of an ultrasonic scanning apparatus according to a preferred embodiment of the present invention;

FIG. 2 is a perspective view showing the ultrasonic scanning apparatus of FIG. 1;

FIGS. 3(a) and 3(b) are conceptual diagram illustrating a process of acquiring a two-dimensional image using the ultrasonic scanning apparatus of FIG. 2; and

FIG. 4 is a flowchart sequentially illustrating a process of obtaining the volume of urine in a bladder using the ultrasonic scanning apparatus according to a preferred embodiment of the present invention.

### DESCRIPTION OF THE PREFERRED EMBODIMENTS

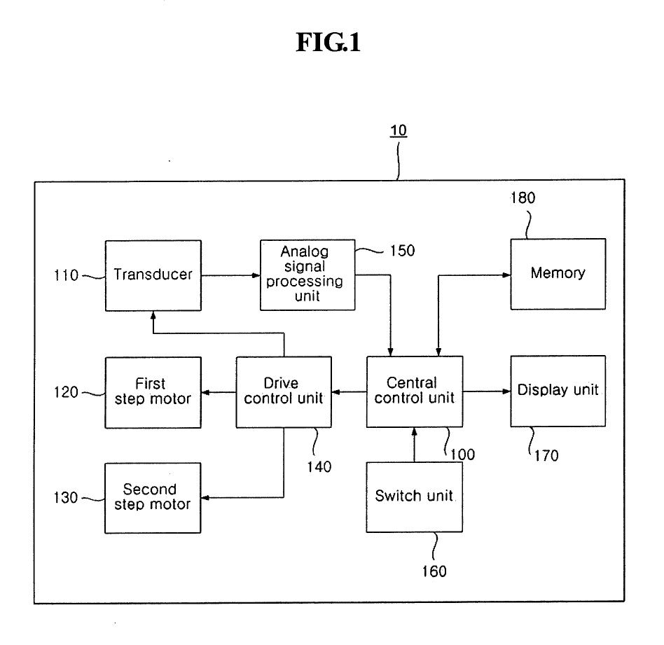

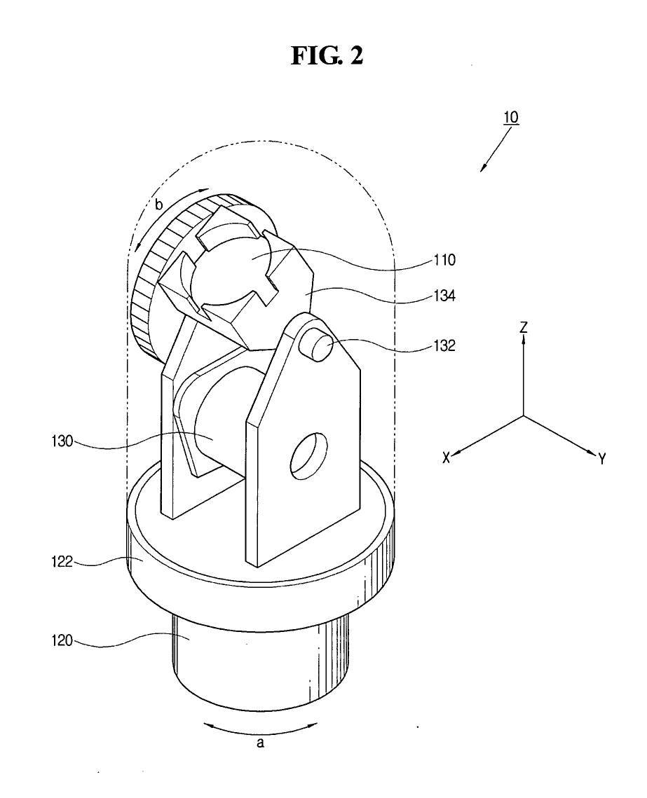

The construction and operation of an ultrasonic scanning apparatus for diagnosing a bladder according to a preferred embodiment of the present invention are described in detail with reference to the accompanying drawings. FIG. 1 is a block diagram schematically showing the internal construction of an ultrasonic scanning apparatus according to a preferred embodiment of the present invention, and FIG. 2 is a perspective view showing the ultrasonic scanning apparatus of FIG. 1.

Referring to FIG. 1, the ultrasonic scanning apparatus 10 according to the preferred embodiment of the present invention includes a central control unit 100 for controlling the overall operation of the apparatus, a transducer 110, a first stepping motor 120, a second stepping motor 130, a drive control unit 140, an analog signal processing unit 150, a switch unit 160, memory 180, and a display unit 170. The respective components of the above-described ultrasonic scanning apparatus 10 are described in detail below.

The transducer 110 is a device that emits ultrasonic signals and receives ultrasonic signals reflected from the internal organs of a human body, and transmits the received analog signals to the analog signal processing unit 150. The transducer 110 of the ultrasonic scanning apparatus for diagnosing a bladder according to the present invention receives ultrasonic signals reflected from urine in the bladder.

The analog signal processing unit 150 converts the analog signals, which are transmitted from the transducer 110, into digital signals, and transmits the digital signals to the central control unit 100.

The central control unit 100 calculates the volume of urine in the bladder, which is an examination object, using the signals transmitted from the analog signal processing unit 150, and outputs the ultrasonic image of the bladder, which is an image related to the specific plane of the bladder, to the display unit 170. The display unit 170 displays the image, which is transmitted from the central control unit, on a screen along with the volume value of urine in the bladder.

As shown in FIG. 2, a rotational support 122 is connected to the first stepping motor 120. A second stepping motor 130 is mounted on the rotational support 122 and rotates along with the rotational support 122. The second stepping motor 130 is connected with a transducer support 134 including a rotational axis 132. A transducer 110 is installed in the transducer support 134.

The central control unit 100 transmits drive control signals to the drive control unit 140 in response to a request signal received from the switch unit 160, and the drive control unit 140 controls the motion of the first and second stepping motors 120 and 130 in response to the drive control signals, so that the ultrasonic image of the bladder can be captured through the rotation of the transducer 110.

The second stepping motor 130 rotates by a predetermined angle in a yz plane, and the rotational axis 132 and the transducer support 134, which are connected to the second stepping motor via a gear, are rotated by the second stepping motor 130. Consequently, the transducer 110 installed in the transducer support 134 rotates in a second direction in the yz plane.

Meanwhile, the rotational support 122, on which the second stepping motor 130 is mounted, is connected to the first stepping motor 120, so that the rotational support 122 also rotates by a predetermined angle in a first direction as the first stepping motor 120 rotates in the first direction. As a result, the first stepping motor 120 and the second stepping motor 130 rotate sequentially in the first direction and the second direction, so that ultrasonic waves are emitted in the form of a cone, for which the transducer 110 is the vertex, therefore measurement is performed.

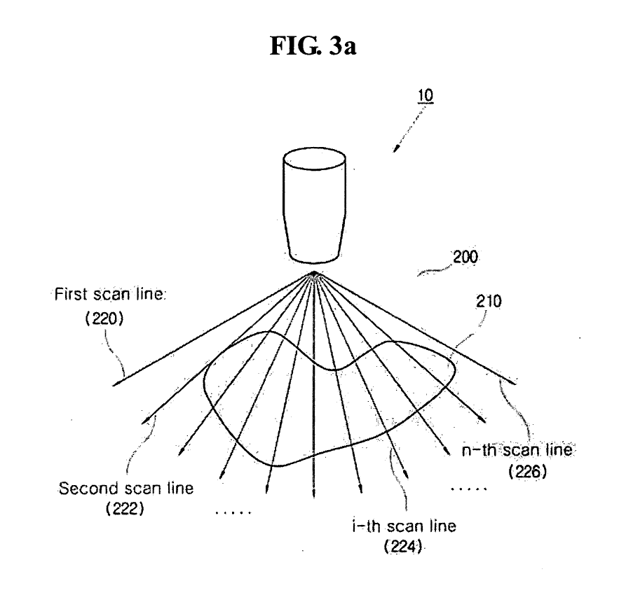

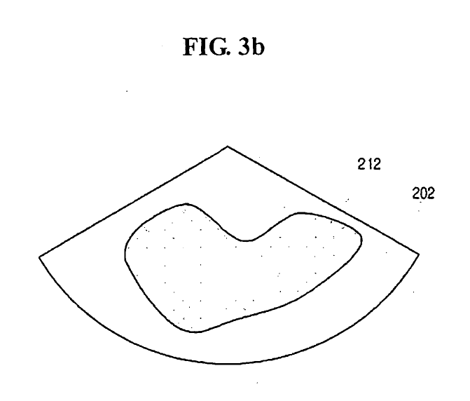

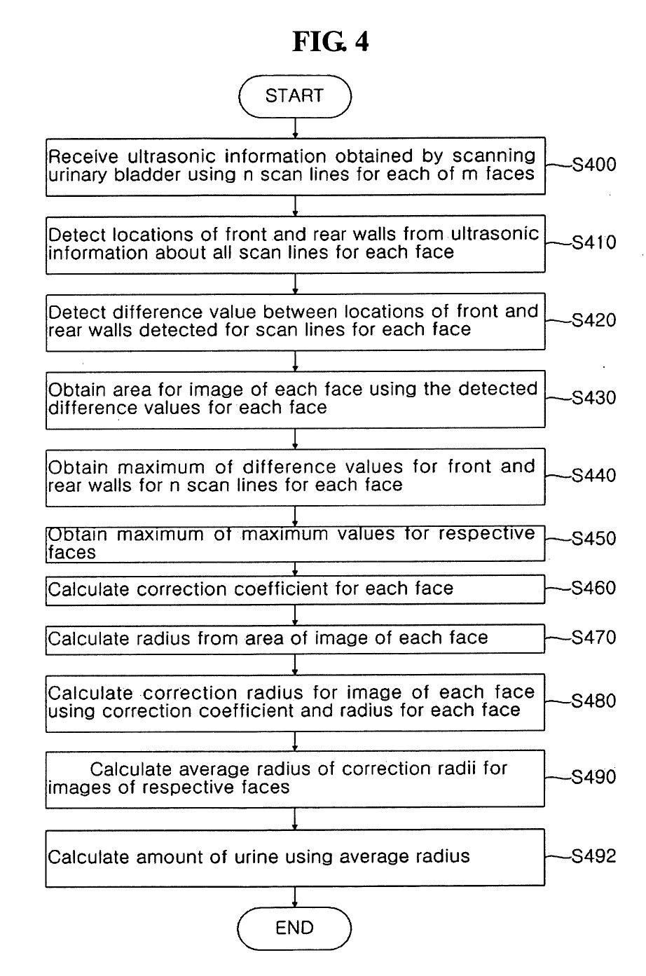

In order to measure the amount of urine in the bladder, the ultrasonic scanning apparatus 10 having the above-described construction, as shown in FIG. 3(a), is located on an abdomen over the bladder 210 of a patient, and sequentially detects respective ultrasonic signals for n scan lines, that is, a first scan line 220, a second scan line, . . . , an ith scan line 224, . . . , an nth scan line 226 while the second stepping motor 130 is rotated by the predetermined angle along a single plane in a state in which the first stepping motor 120 is fixed. After detecting n ultrasonic signals, the central control unit 100, as shown in FIG. 3(b), generates a two-dimensional image by processing ultrasonic signals for each of the planes, and displays the image on the display unit 170. FIG. 3(b) is a diagram showing the image output to the display unit 170, in which urine 212 in the bladder 210 is displayed while being separated from organs 202 around the bladder 210.

Thereafter, ultrasonic signals for n scan lines for the corresponding plane are sequentially detected again while rotating the second stepping motor 130 after rotating the first stepping motor 120 by the predetermined angle and then fixing it. The above described process is repeated, and thus ultrasonic signals for n scan lines for each of m planes are detected. Accordingly, a three-dimensional image can be generated using two-dimensional images that are respectively related to m planes.

A method of the central control unit 100 of the ultrasonic scanning apparatus 10 measuring the amount of urine in a bladder using ultrasonic signals according to a preferred embodiment of the present invention is described in detail below.

First, the transducer 110 of the ultrasonic scanning apparatus scans a bladder, which is a diagnosis object, along n scan lines for each of m planes and receives ultrasonic information obtained through the scanning at step S400. Ultrasonic information about n scan lines for a single plane is received and, as a result, pieces of ultrasonic information for the m planes are received. The number of planes to be scanned and the number of scan lines for a single plane may be determined according to the region and size of the diagnosis object. In the case of measuring a bladder, the number of scan lines and the number of images may be determined such that the overall region of the bladder can be sufficiently included. For example, in the case of scanning the bladder, the overall region of the bladder can be sufficiently included using about 67 lines if the angle between lines for forming a single image is 1.8°.

Thereafter, the locations of front and rear walls are detected using ultrasonic information about scan lines constituting each plane at step S410, difference values Depth[1], Depth[2], . . . , Depth[n] corresponding the differences between the locations of the detected front and rear walls for the respective scan lines for the corresponding plane are obtained at step S420, and areas Area[1], Area[2], . . . , Area[m] for respective planes are obtained using the difference values for the scan lines constituting each plane at step S430. In this case, the method of obtaining an area for each plane using difference values corresponding to the differences between the locations of the front and rear walls of the respective scan line may be implemented in various ways. As an example, a method of obtaining the entire area for all lines having respective rear walls after obtaining an area for a sector, which is formed by a single scan line using the rotational angle of the second stepping motor 130, may be implemented. As another example, a method of obtaining the entire area for all lines having respective rear walls after obtaining an area for a trapezoid, which is formed by the two front walls and two rear wall of two scan lines, may be implemented.

Thereafter, in the case of obtaining a three-dimensional volume using a plurality of two-dimensional images, an amount smaller than an actual amount is calculated and, thus, an error occurs if scanning is performed in a state in which the center of a first rotational axis moves from the center of the bladder. Accordingly, numerical correction is performed to reduce such error and accurately measure the amount of urine in the bladder.

Thereafter, the maximum values bladderDepth[1], bladderDepth[2], . . . , bladderDepth[m] of the respective planes, each of which is the maximum of difference values corresponding to the differences between the locations of front and rear walls for the n scan lines constituting each image, are obtained at step S440, and the greatest ‘MaxbladderDepth’ of the maximum values of the respective planes is obtained at step S450.

Thereafter, at step S460, the correction coefficients ComFactor[1], ComFactor[2], . . . , ComFactor[i], and ComFactor[m] for the respective planes are obtained using the following equation 1:

$$ComFactor [ i ] = \frac{MaxBladderDepth}{BladderDepth [ i ]} \qquad ( 1 )$$

Thereafter, given the assumption that an image for each plane is a circle, radii r[l], r[2], . . . , r[i], and r[m] are obtained using areas Area[1], Area[2], . . . , Area[m] for the calculated images for the respective plane at step S470.

Thereafter, at step S480, corrected radii ComR[1], ComR[2], . . . , ComR[i], and ComR[m] with respect to the correction coefficients and the radii for the respective images are obtained using the following Equation 2:
ComR[i]=ComFactor[i]×r[i] (2).

An average radius ‘AverageR’, which is the average value of the calculated corrected radii for the images of the respective plane, is obtained at step S490. Thereafter, given the assumption that the complete bladder is a sphere, the total volume V of urine in the bladder by applying the average radius to the following Equation 3 is obtained at step S492.

$$V = \frac{4}{3} π Average {R}^{3} \qquad ( 3 )$$

From the above-described process, the ultrasonic scanning apparatus for diagnosing a bladder according to the present invention can accurately detect the amount of urine in the bladder.

As described above, the present invention provides a single transducer and two stepping motors, each having a rotational axis, so that it can provide an ultrasonic scanning apparatus that has a small size and a light weight and, at the same time, provides ultrasonic information about a three-dimensional image.

Furthermore, the ultrasonic scanning apparatus of the present invention collects the ultrasonic information while automatically rotating the two stepping motors, so that it can collect all pieces of ultrasonic information within a region defined in a cone shape having a vertex at the location at which the ultrasonic scanning apparatus is disposed. As a result, although each of the conventional apparatuses measures the amount of urine in a urinary bladder using only ultrasonic information about two planes, and thus generates inaccurate data, the apparatus of the present invention can very accurately measure the amount of urine using ultrasonic information about a plurality of planes that are spaced apart from each other and exist in an angle of 360°.

In particular, the apparatus of the present invention uses correction coefficients obtained by calculating the degree to which a first detection location is moved from the center of the urinary bladder, so that it can perform accurate measurement even when the detection location is moved from the center of the urinary bladder.

Although the present invention has been described in detail in conjunction with the preferred embodiment, the present invention is described only for illustrative purposes and is not limited thereto. Those skilled in the art will appreciate that various modifications and applications, which are not described above, are possible within a range that does not change the substantial characteristics of the present invention. For example, in the present embodiment, the method of obtaining an area for a corresponding plane using the rotational angles of the first stepping motor and the second stepping motor and ultrasonic information about the respective scan lines may be modified and implemented in various ways to improve scanning performance. Furthermore, it should be appreciated that the differences regarding the modifications and the applications are included in the scope of the present invention, which is defined by the accompanying claims.

What is claimed is:

1. An ultrasonic scanning apparatus for diagnosing a bladder, comprising:
a transducer for emitting ultrasonic signals for respective scan lines, and receiving ultrasonic signals reflected from an object;
a transducer support configured such that the transducer is fixedly installed therein;
an analog signal processing unit for converting the ultrasonic signals, which are transmitted from the transducer, into digital signals;
a display unit for outputting specific image signals;
a central control unit for performing image processing on the digital ultrasonic signals transmitted from the analog signal processing unit, outputting results of the processing to the display unit, and controlling overall operation of the apparatus;
a first stepping motor for rotating in a first direction;
a second stepping motor for rotating in a second direction; and
a drive control unit for controlling operation of the first and second stepping motors in response to drive control signals provided from the central control unit;
wherein the transducer support rotates in the first direction as the first stepping motor rotates, the transducer support rotates in the second direction as the second stepping motor rotates, and the central control unit calculates an amount of urine in the bladder using a plurality of pieces of ultrasonic information about n scan lines for each of m planes of the bladder, which are sequentially received from the analog signal processing unit.

2. The ultrasonic scanning apparatus as set forth in claim 1, wherein the plurality of the ultrasonic information about n scan lines for each of m planes of the bladder are received through a process of receiving ultrasonic information about a predetermined number of scan lines for each plane of the bladder while rotating the second stepping motor, and rotates the first stepping motor by a predetermined angle.

3. The ultrasonic scanning apparatus as set forth in claim 1, wherein the central control unit detects locations of front and rear walls using the ultrasonic information about n scan lines for each of m planes, obtains difference values corresponding to differences between the detected locations of the front and rear walls for the scan lines, obtains an area for an image of a corresponding plane using the difference values, obtains correction coefficients for respective planes, calculates radii using areas for images of respective planes, and calculating corrected radii by applying the correction coefficients to the calculated radii, obtains an average radius of the corrected radii for the respective planes, and obtains a total volume using the average radius.

4. The ultrasonic scanning apparatus as set forth in claim 3, wherein the central control unit detects a maximum of the difference values for the respective scan lines for each plane, obtains a greatest of the maximum values for the respective planes, and obtains a correction coefficient for each plane using a ratio of the maximum value for each plane to the greatest of the maximum values.

5. An ultrasonic diagnosis method, the ultrasonic diagnosis method measuring an amount of urine in a bladder by sequentially receiving ultrasonic information about n scan lines for each of m planes of the bladder from a transducer of an ultrasonic scanning apparatus, the ultrasonic diagnosis method comprising the steps of:
(a) detecting locations of front and rear walls using the ultrasonic information about n scan lines for each of m planes;
(b) obtaining difference values between the detected locations of the front and rear walls for the respective scan lines;
(c) obtaining an area for an image of a corresponding plane using the difference values;
(d) obtaining correction coefficients for respective planes;
(e) calculating radii using areas for images of respective planes, and calculating corrected radii by applying the correction coefficients to the calculated radii;
(f) obtaining an average radius of the corrected radii for the respective planes; and
(g) obtaining a total volume using the average radius.

6. The ultrasonic diagnosis method as set forth in claim 5, wherein the step (d) comprises:
(d-1) detecting a maximum of the difference values for the respective scan lines for each plane;
(d-2) obtaining a greatest of the maximum values for the respective planes; and
(d-3) obtaining a correction coefficient for each plane using a ratio of the maximum value for each plane to the greatest of the maximum values.

7. The ultrasonic diagnosis method as set forth in claim 6, wherein the corrected coefficient of the step (d-3) is calculated using the following equation:
$$ComFactor [ i ] = \frac{MaxBladderDepth}{BladderDepth [ i ]}$$
where ComFactor[i] is a corrected coefficient for an ith plane, bladderDepth[i] is the maximum of the difference values between locations of front and rear walls for scan lines for the ith plane, and MaxBladderDepth is the greatest of the maximum values of the respective planes.
